(19) United States
(12) Patent Application Publication
(10) Pub. No.: US 2023/0116904 A1
(43) Pub. Date: Apr. 13, 2023

(54) SELECTING A CELL LINE FOR AN ASSAY

(71) Applicant: BenevolentAI Technology Limited, London (GB)

(72) Inventors: Aaron SIM, London (GB); Francesca MULAS, London (GB); Poojitha OJAMIES, London (GB); Craig GLASTONBURY, Reading (GB); Povilas NORVAISAS, London (GB); Paidi CREED, London (GB)

(73) Assignee: BenevolentAI Technology Limited, London (GB)

(21) Appl. No.: 17/904,911

(22) PCT Filed: Feb. 12, 2021
(86) PCT No.: PCT/GB2021/050342

§ 371 Date: Aug. 24, 2022

(30) Foreign Application Priority Data
Feb. 24, 2020 (GB) 2002586.2

(51) Int. Cl. G16B 20/00; G16B 40/30

(52) U.S. Cl. CPC G16B 20/00; G16B 40/30

(57) ABSTRACT

A computer-implemented method and a system of selecting a cell line for an assay. The computer-implemented method and system encode data, which is comprised of one or more features, as one or more latent variables. The one or more features encoded in the one or more latent variables are identified and mapped to cell lines based on the one or more features. A relevance of one or more targets to each of one or more of the one or more latent variables is determined and the one or more targets to the cell lines are matched via the one or more latent variables.

The present application relates to systems and methods for selecting cell lines to be used in assays for the purpose of drug testing. The presently disclosed techniques find particular application in the fields of biochemistry and drug discovery where a cell line with certain characteristics may be required to test a drug target hypothesis.

### BACKGROUND

In the field of drug discovery, there is a need to identify suitable cell lines to be used in assays for drug testing. Cell lines that are likely to respond in a desired clinical way to a drug under test are required to test the drug target hypothesis. Traditionally, the identification of suitable cell lines can be achieved using ground-up modelling to predict which cell lines are likely to produce the desired clinical effect. However, this approach can be costly and time consuming, and risks the introduction of assumptions that distort the predicted results.

Accordingly, there is a need for an improved technique for identifying suitable cell lines for assays that improves the accuracy of predicted results and does not introduce unwanted assumptions.

The embodiments described below are not limited to implementations which solve any or all of the disadvantages of the known approaches described above.

### SUMMARY

This Summary is provided to introduce a selection of concepts in a simplified form that are further described below in the Detailed Description. This Summary is not intended to identify key features or essential features of the claimed subject matter, nor is it intended to be used to determine the scope of the claimed subject matter.

The present disclosure provides a method and system for selecting cell lines to be used in assays for the purpose of drug testing. In general, the invention receives, as input, targets for drugs and delivers, as output, a list of cell lines. The list of cell lines may be ranked or otherwise categorised to indicate their predicted utility in assays, or have some other indication of their propensity score for each input target. To deliver the output, an input set is mapped to a series of latent variables to identify one or more features encoded in the one or more latent variables. Once the mapping of the one or more latent variables to the cell lines has been performed, the one or more latent variables may be examined to determine which specific feature or group of features are encoded in which latent variables. In turn, the targets of drugs may be represented by specific features or groups of features known to interact with or otherwise be affected by the drugs being considered, in accordance with the mapping of the latent variables to the cell lines.

In a first aspect, the present disclosure provides a computer-implemented method of selecting a cell line for an assay, the method comprising: encoding data, which is comprised of features, as latent variables; identifying one or more features encoded in the one or more latent variables; mapping the latent variables to cell lines based on the one or more features; determining a relevance of one or more targets to each of one or more of the latent variables; and matching the one or more targets to the cell lines via the latent variables.

Optionally, the encoded data comprises at least one of genomics data, transcriptomics data, methylation data, clinical data applied to genes, and biological mechanisms associated with multiple features.

Optionally, encoding the data as latent variables using linear and non-linear machine learning models.

Optionally, encoding the data as latent variables using a matrix factorisation approach.

Optionally, encoding the data as latent variables using a clustering algorithm.

Optionally, associating a biological mechanism with each latent variable based on the one or more features that the latent variable encodes.

Optionally, determining an extent to which each latent variable is associated with a respective biological mechanism.

Optionally, assessing the latent variables for relevance to a disease.

Optionally, determining an extent to which a respective biological mechanism associated with a latent variable is associated with the disease.

Optionally, annotating the latent variables based on relevance to the disease.

Optionally, removing from consideration latent variables not sufficiently relevant to the disease.

Optionally, two or more latent variables represent respective biological mechanisms of the disease.

Optionally, using the latent variables to stratify patients into endotypes.

Optionally, mapping a latent variable to a cell line if one or more features in the cell line sufficiently match the one or more features encoded in the latent variable.

Optionally, assigning a mapping value to each latent variable and cell line pair based on a relevance of the cell line to the latent variable.

Optionally, assigning a mapping value to each latent variable and cell line pair based on a relevance of the cell line to the latent variable, where the relevance of the cell line to the latent variable is based on an extent to which features in the cell line matches the one or more features encoded in the latent variable.

Optionally, determining a relevance score of each target to each latent variable based on the extent to which the target regulates one or more of the genes encoded in the latent variable.

Optionally, regulation of the genes encoded in the latent variable comprise an indirect association with the latent variable genes.

Optionally, annotating a respective latent variable with targets that sufficiently regulate one or more of the features encoded in the respective latent variable.

Optionally, matching targets to cell lines by comparing the mapping of the latent variables to the cell lines and the relevance of the targets to the latent variables.

Optionally, assigning a mapping value to each latent variable and cell line pair based on a relevance of the cell line to the latent variable; determining a relevance score of each target to each latent variable based on the extent to which the target regulates one or more of the genes encoded in the latent variable. For each latent variable, determining a metric between each target and each cell line based on: the mapping value of the latent variable and the cell line; and the relevance score of the target and the latent variable.

Optionally, determining a metric between each target and each cell line, where the metric is also based on the relevance of the latent variable to the disease.

Optionally, outputting a ranked list of cell lines for each target based on the metrics.

Optionally, the one or more features comprise one or more genes, methylation sites, and genetic variants.

Optionally, the one or more features of the cell line comprise gene expression.

In a second aspect, the present disclosure provides a computer-readable medium storing code that, when executed by a computer, causes the computer to perform the computer-implemented method according to any one of the features, steps, process(es) of the first aspect, combinations thereof, modifications thereto, and/or as herein described.

In a third aspect, the present disclosure provides a system for selecting a cell line for an assay, the system comprising: an encoder configured to encode data as latent variables; an interpretation module configured to identify the one or more features encoded in the latent variables; a cell line mapping module configured to map the latent variables to cell lines; a target relevance module configured to determine a relevance of one or more targets to each of one or more of the latent variables; and a matching module configured to match the one or more targets to the cell lines via the latent variables.

Optionally, in the third aspect, the system for selecting a cell line for an assay performs the computer-implemented method according to any one of the features, steps, process(es) of the first aspect, combinations thereof, modifications thereto, and/or as herein described.

The computer-implemented methods described herein may be performed by software in machine readable form on a tangible storage medium e.g. in the form of a computer program comprising computer program code means adapted to perform all the steps of any of the methods described herein when the program is run on a computer and where the computer program may be embodied on a computer readable medium. Examples of tangible (or non-transitory) storage media include disks, thumb drives, memory cards etc. and do not include propagated signals. The software can be suitable for execution on a parallel processor or a serial processor such that the method steps may be carried out in any suitable order, or simultaneously.

This application acknowledges that firmware and software can be valuable, separately tradable commodities. It is intended to encompass software, which runs on or controls “dumb” or standard hardware, to carry out the desired functions. It is also intended to encompass software which “describes” or defines the configuration of hardware, such as HDL (hardware description language) software, as is used for designing silicon chips, or for configuring universal programmable chips, to carry out desired functions.

The preferred features may be combined as appropriate, as would be apparent to a skilled person, and may be combined with any of the aspects of the invention.

### BRIEF DESCRIPTION OF THE DRAWINGS

Embodiments of the invention will be described, by way of example, with reference to the following drawings, in which:

FIG. 1 is a block diagram of a system for selecting a cell line for an assay according to an embodiment of the invention;

FIG. 2 is a flow chart of a method that may be carried out by the system of FIG. 1 according to an embodiment of the invention;

FIG. 3 is a block diagram of an example data source that may be used by the system;

FIG. 4 is a schematic diagram of an encoder of the system showing optional features of the encoder and an output of the encoder;

FIG. 5 is a schematic diagram of an autoencoder according to some embodiments of the invention;

FIG. 6 is a block diagram of an example processed output of the encoder;

FIG. 7 is a schematic diagram showing matching of targets to cell lines by the system; and

FIG. 8 is a block diagram of a computer suitable for implementing embodiments of the invention.

Common reference numerals are used throughout the figures to indicate similar features.

### DETAILED DESCRIPTION

Embodiments of the present invention are described below by way of example only. These examples represent the best ways of putting the invention into practice that are currently known to the Applicant although they are not the only ways in which this could be achieved. The description sets forth the functions of the example and the sequence of steps for constructing and operating the example. However, the same or equivalent functions and sequences may be accomplished by different examples.

In the fields of biochemistry and drug discovery, the task of developing new treatments for diseases frequently involves drug testing. When a drug is being tested for its clinical effect on a disease, the process starts with a hypothesis that the modification of a biological target by the drug will produce a desired clinical effect. As a result, drug testing is designed to test the underlying hypothesis relating to the clinical effect of the modified target on the disease. This test is therefore part of a wider approach known as a target validation framework which relies on prior knowledge of which drugs are known to affect or modify the targets being considered. In this document, the terms ‘biological target’ and ‘target’ are used interchangeably and refer to biological molecules that are involved in a disease mechanism or disease pathway with which drugs or other therapeutic agents (e.g. small molecules, ligands, macromolecules, biologics, and antibodies) may bind. For example, biological targets may comprise genes, ribonucleic acid (RNA) and other genomic entities.

In order to test a hypothesis that a modified target will have a desired clinical effect, a cell line needs to be selected that is relevant to the disease. However, for any given disease there may be multiple subgroups of the disease, each caused by a different underlying mechanism. Since a drug under test will usually only have a desired clinical effect on one of the disease mechanisms, a model is required that can differentiate between disease subgroups and successfully predict which cell lines are likely to respond to the drug. With such a model, suitable cell lines can be selected for use in an assay to verify the efficacy of a drug for treating a subgroup of a disease.

Furthermore, the effect of a modified target on a cell line is usually not as simple as upregulating or downregulating the production of a single gene or protein producing a disease state. Often, the effect of a modified target may involve a cascading effect of downstream regulation in a network of genes and proteins in a disease pathway. As a result, a model is required that can accommodate the true impact of a modified target including downstream effects and still select a suitable cell line for an assay.

The inventor has appreciated that there is a need for a system that can accurately select cell lines to be used in assays for validating target hypotheses whilst handling complexity and not introducing assumptions.

Accordingly, the present disclosure relates to an approach in which molecular and/or clinical patient data are encoded as latent variables. This type of encoding allows groupings of related biological features such as genes to be extracted as latent variables from the data without introducing assumptions. Each latent variable represents a different grouping of related biological features such as a grouping of genes and other features that together may represent an underlying biological mechanism. As a result, encoding the data as latent variables provides a way of separating different mechanisms of the same disease into separate clusters so that they can be considered independently of each other. As such, it will be appreciated that the latent variables may be used to stratify patients into endotypes.

The biological meanings such as disease mechanisms that the latent variables represent can be identified by determining one or more features that each latent variable encodes, where the latent variable features may correspond to one or more genes, methylation sites, genetic variants, and/or other measurable biological entities. This enables the latent variables to provide a useful connection for matching targets with suitable cell lines because targets that are relevant to a latent variable can be matched with cell lines that map to the latent variable, as will be described further below.

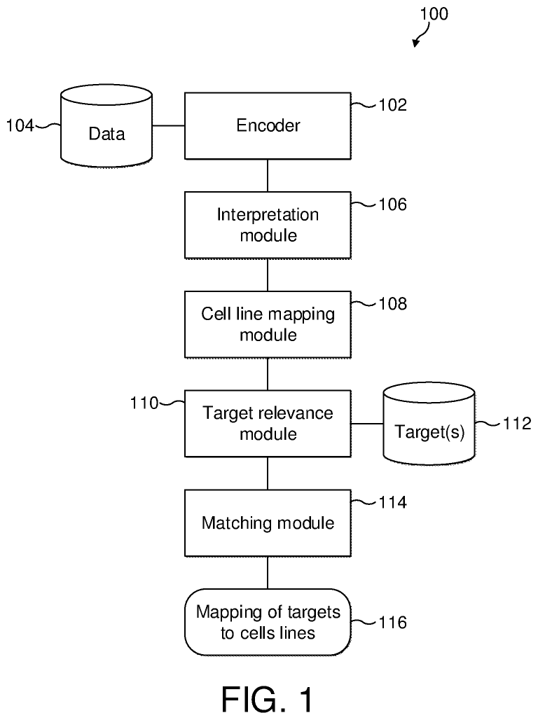

FIG. 1 shows a system 100 for selecting a cell line for an assay according to an embodiment of the invention. The system 100 comprises an encoder 102 configured to encode data 104 as latent variables. The data 104 relates to molecular and clinical patient data, for example of patients having a disease of interest. The latent variables represent high dimensional data in a compressed form and may be generated using techniques including machine learning methods such as the use of an autoencoder.

The system 100 further comprises an interpretation module 106 configured to identify one or more features encoded in the latent variables, an example would be a latent variable that represents one or more genes. This identification of the one or more features encoded in the latent variables forms the basis of interpreting the latent variables, in particular of identifying a biological meaning such as a biological mechanism represented by each latent variable. In this document, the terms ‘biological mechanism’ and ‘mechanism’ are used interchangeably and refer to a collection of processes that work together to produce a biological result such as a physiological state or a pathological or disease state. An example of a biological mechanism is a disease mechanism which refers to a collection of processes that work together to produce a pathological or disease state.

The interpretation module 106 may be configured to use statistical tests to try to assign biological meaning to the latent variables. The process of interpretation may involve the use of attribution methods to associate known features such as genes, metabolic networks and disease processes to each latent variable, and may additionally or alternatively involve the utilisation of any labels that may exist in the input data. Additional processing or analysis of the latent variables may take place at this time such as filtering on positive silhouette scores, and the latent variables may be annotated with, for example, marker genes, or otherwise enriched.

In particular, the association of biological meaning to the latent variables may comprise associating a biological mechanism with each latent variable based on the features that the latent variable encodes. The features may be genes that the latent variable encodes. In this case, the interpretation module 106 may be configured to determine an extent to which each latent variable is associated with biological mechanism. This provides a measure of confidence in the association between a latent variable and a biological mechanism, where a high confidence relates to a high likelihood that there is an association and a low confidence relates to a low likelihood that there is an association and a possibility that any apparent association may be a result of noisy data. For the purpose of ignoring noise, the interpretation module 106 may be configured to remove from consideration latent variables not sufficiently associated with a biological mechanism so that latent variables from which a biological meaning cannot be inferred are ignored by later modules of the system 100.

As well as assigning biological meaning to latent variables, the interpretation module 106 may be configured to assess the latent variables for their relevance to a disease of interest. This step can be used to identify which latent variables are relevant to the disease and to remove from consideration any other latent variables that might have other biological meanings. For example, some of the latent variables may represent biological processes such as the aging process that are present in all biological subjects regardless of whether they have the disease, but these latent variables are unlikely to be useful for validating targets for treating the disease. As such, the interpretation module 106 may be configured to determine an extent to which a biological mechanism associated with a latent variable is associated with the disease, and may additionally or alternatively be configured to annotate the latent variable based on its relevance to the disease. A biological mechanism that is associated with a disease may comprise a disease mechanism, or at least a mechanism that contributes to the disease state by causing a pathological process, or alternatively a mechanism that has been modified as a result of the disease state.

The interpretation module 106 may be configured to score latent variables for relevance to a disease. This may be achieved by applying descriptive statistics to the latent variables to produce scores that correspond to a biological property related to the disease. The scores may be aggregated over so that each latent variable is assigned a single score for relevance to the disease. The latent variables may be ranked using the scores and the latent variables having the highest scores indicating the highest relevance to the disease may be used for target validation.

Referring to FIG. 1, the system 100 comprises a cell line mapping module 108 configured to map the latent variables to available cell lines with which assay testing may be performed. The mapping is on the basis of feature correspondence between the cell lines and features encoded in the latent variables. If the pattern associated with features in the cell line sufficiently match one or more features encoded in the latent variable, then a latent variable is mapped to a cell line. For example, if a pattern of gene expression in a cell line sufficiently matches the one encoded in a latent variable, this indicates that the mechanism associated with the latent variable is active in the cell line, by way of the mapping.

In general, a feature describes a measurable property of an object that could be analysed. In the field of drug discovery, the analysable object may be a biological sample derived from a patient or an assay, in which case, certain biological properties (or features) have been measured. Depending on the method of measurement used, one or more features may be expression of genes (transcriptomics data), presence of methylation (methylation data) or presence of genetic variants (genomics data).

In addition, the patient may also be seen as an object with measurable features (e.g. age, body mass, gait speed, CRP levels, etc.) (including clinical data or metadata). As such features herein described may include more than just specific types of genes or genomic features but any features, for example, to perform patient endotyping. The one or more features pertain to observation or objects whether it may be patient, sample, assay or otherwise describing the particular object.

In particular, the one or more features encoded in the latent variable may be derivable from assays that screen the cell line pertaining these features, include, by way of example, but not limited to a gene. On the other hand, features in the cell line pertain to other screen-able characteristics of the cell line, which include, by way of example, but not limited to gene expression or pattern of gene expression.

The cell line mapping module 108 may be configured to assign a mapping value to each latent variable and cell line pair based on a relevance of the cell line to the latent variable. This mapping value may represent a measure of confidence that a latent variable maps to a cell line, and/or may be based on an extent to which features in the cell line matches the one or more features encoded in the latent variable. For example, the features in the cell line may be the expression of a given gene, which are encoded in a low dimensional latent variable (a latent). In this example, the cell line mapping module 108 may achieve the mapping using a number of techniques such as single sample gene set enrichment analysis to quantify the enrichment of a latent variable's gene set in a cell line. Other similarity metrics may also be employed.

The system 100 comprises a target relevance module 110 configured to determine a relevance of one or more targets 112 to the latent variables. The targets 112 are provided as an input to the system so that cell lines to be used in assays can be identified by the system 100 in order to validate whether the targets have a desired clinical effect on a disease when modified by a drug. Each target 112 is scored, ranked or weighted according to its respective relevance to each of the latent variables. For example, the target relevance module 110 may be configured to determine a relevance score of each target to each latent variable based on the extent to which the target regulates one or more of the genes encoded in the latent variable. This may be performed using any suitable analytical method, for instance a binary true-false metric for the presence of the target encoded in the latent variable, or a weighted score based on other analytical data.

The target relevance module 110 may additionally or alternatively be configured to annotate each latent variable with targets that sufficiently regulate one or more of the features it encodes. Each feature may represent a gene expression value or any other genomic entity that is relevant and measurable. It is to be appreciated that the determination of relevance of targets to latent variables may take place before, simultaneously with or after the mapping of latent variables to cell lines.

If a target 112 regulates multiple genes encoded in the same latent variable, it may be highly relevant to that latent variable and could therefore be especially effective to treat a patient subgroup with the associated underlying disease mechanism. In some cases, a target might even regulate multiple genes in more than one latent variable, and therefore may indicate a drug that would be effective for treating multiple mechanisms of the disease.

The notion of target regulating genes in the context of this application may potentially be in the form of a direct or an indirect association. In case of the indirect association, latent variable genes may be associated in ways such as through the use of mutations, expression quantitative trait loci (eQTL), and the like, as to identify complex patterns of association between the target and the latent variable.

Finally, the system also comprises a matching module 114 configured to match targets to cell lines via the latent variables, and to thereby output a mapping 116 of targets to cell lines. The mapping may be performed by a comparison of the mapping of the latent variables to the cell lines and the relevance of the targets to the latent variables. For example, the matching module 114 may be configured to determine, for each latent variable, a metric between each target and each cell line. The metric may be based on a value or score indicating a relevance of the cell line to the latent variable and on a value or score indicating the relevance of the target to the latent variable. The metric may also be based on the relevance of the latent variable to a disease of interest. The metrics may for example be computed by summing, multiplying or otherwise aggregating relevant values or scores between the targets and the latent variables and between the latent variables and the cell lines.

Suitably, the matching module 114 may be configured to output a ranked list of cell lines for each target based on the metrics, a weighting between cell lines and targets, or any other form of score or metric indicating relevance. Other suitable outputs may for example include a table matching targets to suitable cell lines.

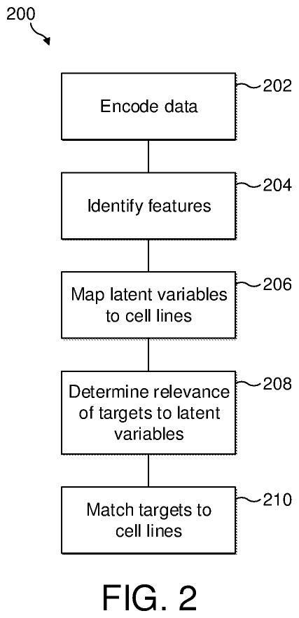

With reference to FIG. 2, the present disclosure extends to a computer-implemented method 200 of selecting a cell line for an assay. The method 200 comprises encoding 202 data as latent variables; identifying 204 one or more features encoded in the latent variables, where the identified one or more features may be genes; mapping 206 the latent variables to cell lines; determining 208 a relevance of one or more targets to each of one or more of the latent variables; and matching 210 the one or more targets to the cell lines via the latent variables.

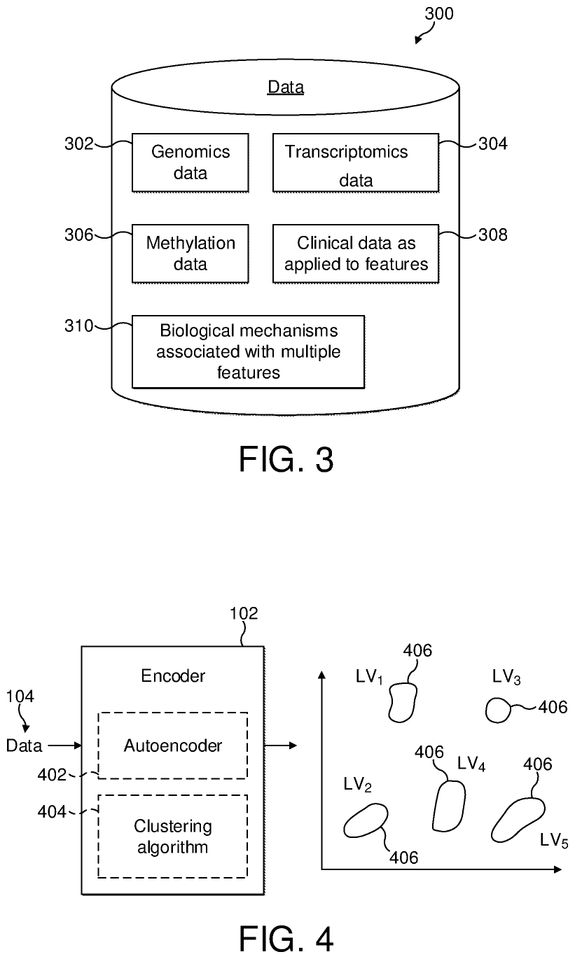

Referring to FIG. 3, an exemplary set of data 300 provides an example of the data 104 that is encoded as latent variables by the encoder 102. The data 300 relates to molecular and clinical patient data, for example from a group of patients all having the same disease. The data 300 comprises genomics data 302, transcriptomics data 304, methylation data 306, clinical data as applied to features 308 and biological mechanisms associated with multiple features 310. The multiple features may be multiple genes, In other example sets of data there may be similar or other extensive measurements of the presence or expression of proteins and genes for example from microarrays, RNA sequencing data, genetic sequencing data, genotyping data, copy number variation, clinical data such as patient age or gender, and longitudinal data representing clinical outcomes. Data sets may also include metadata. For example, patient genetic sequence data could be accompanied by patient survival time data from a follow up study. A suitable data set may, for example, be based on data from approximately 100 patients although this figure is non-limiting and not intended as a guide.

In suitable examples, the encoder 102 may be configured to encode the data 104 as latent variables using linear and non-linear machine learning models. Supervised and unsupervised machine learning techniques may be used. Referring to FIG. 4, the encoder 102 may optionally include, by way of example, but not limited to autoencoders 402, matrix factorisation approaches (not shown), and/or clustering algorithms 404 for encoding the data 104 as latent variables. A schematic representation of latent variables 406 is shown in FIG. 4 to illustrate the separation of different mechanisms into separate clusters that can be considered independently of each other.

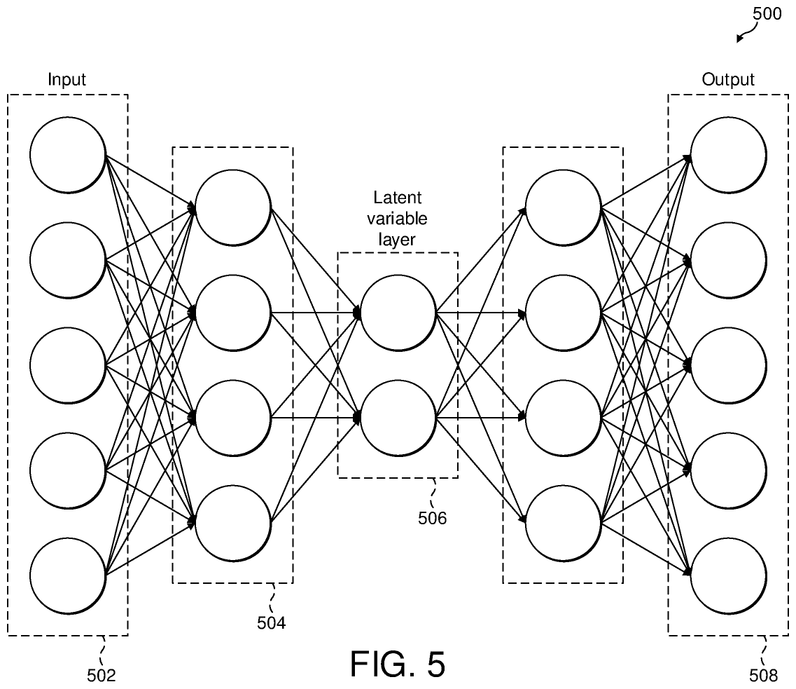

FIG. 5 shows an example of an autoencoder 500 with which latent variables may be generated. In this example, an input vector 502 is passed through a neural network of one or more layers of hidden nodes 504 to an intermediate layer with fewer nodes than the input—that is with a dimensionality reduction 506. These nodes are connected to additional nodes in additional layers to a series of output nodes 508 of the same dimensionality as the input layer. Such a system may be trained to reconstruct input data at the output, resulting in compact, lower dimensional representations of different inputs in the intermediate latent variable layer 506.

As an alternative, a variational autoencoder may be used that additionally encodes a mean and standard deviation vector, which is sampled at the latent variable stage before being decoded back to the original input.

Additionally or alternatively, a further method for generating latent variable representations may be using unsupervised machine learning techniques or other clustering algorithms, such as k-means, mixture models, density-based spatial clustering of applications with noise (DBSCAN), or other methods. These methods may be linear or non-linear. These methods may also include various matrix factorisation approaches, such as singular value decomposition or other decompositions, such as non-negative matrix factorization, binary matrix factorization, and/or probabilistic versions of matrix factorisation. It will be appreciated that latent variables may be generated using one of the above methods or a combination of those methods.

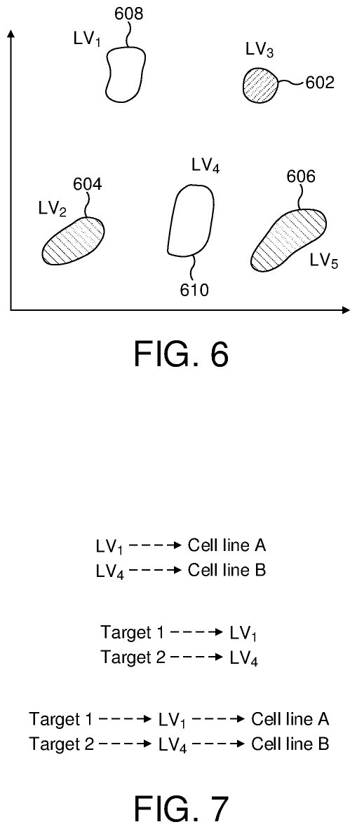

Referring to FIG. 6, a schematic diagram is shown illustrating the interpretation and processing of latent variables for the purpose of identifying latent variables that are relevant to a disease of interest. After identifying biological mechanisms associated with the latent variables, any latent variables such as latent variable LV₃ 602 which are found not to be sufficiently associated with a biological mechanism are taken to be a product of noisy data and may be removed from further consideration. Similarly, any latent variables such as latent variables LV₂ 604 and LV₅ 606 that are found to represent biological mechanisms not related to the disease of interest may also be removed from further consideration. This leaves latent variables such as LV₁ 608 and LV₄ 610 that represent mechanisms that are relevant to the disease of interest and can be used to match targets to available cell lines.

Continuing this example, a schematic overview of a process of matching targets with cell lines is shown in FIG. 7. The latent variables of interest, LV₁ and LV₄, are mapped to cell lines A and B respectively. Separately, targets 1 and 2 are found to be relevant for latent variables LV₁ and LV₄, respectively. By comparing the mapping of latent variables to cell lines with the relevance of targets to latent variables, the targets can be matched to the cell lines via the latent variables. In this case, target 1 is determined to match cell line A and target 2 is determined to match cell line B.

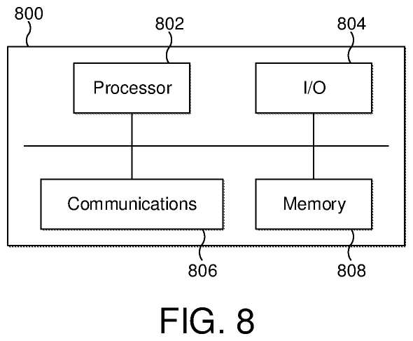

A computer apparatus 800 suitable for implementing methods according to the present invention is shown in FIG. 8. The apparatus 800 comprises a processor 802, an input-output device 804, a communications portal 806 and computer memory 808. The memory 808 may store code that, when executed by the processor 802, causes the apparatus 800 to perform the method 200 shown in FIG. 2.

Embodiments according to the present disclosure are associated with various advantages. The use of latent variables for selecting cell lines provides a more accurate selection of cell lines than other methods that match targets to cell lines via single gene expression. The matching of targets to cell lines via single gene expression is associated with considerable noisy data, and it may be that a target gene is highly expressed in all or most cell lines, so it is not sufficiently selective.

Furthermore, if the approach of the present disclosure is used to successfully predict a group of cell lines that will show a desired clinical effect from the modification of a target and to predict another group of cell lines that will not show the desired clinical effect from the modification of the target, then this verifies not only that modification of the target has the desired clinical effect (i.e. the drug being used treats the disease) but also the underlying mechanism of the disease in those cell lines where the desired clinical effect is shown.

Finally, since cell lines were successfully predicted based on a mechanistic hypothesis, the hypothesis is that the drug used will be efficacious in pre-clinical assays and future clinical trials.

In the embodiment described above the server may comprise a single server or network of servers. In some examples the functionality of the server may be provided by a network of servers distributed across a geographical area, such as a worldwide distributed network of servers, and a user may be connected to an appropriate one of the network of servers based upon a user location.

The above description discusses embodiments of the invention with reference to a single user for clarity. It will be understood that in practice the system may be shared by a plurality of users, and possibly by a very large number of users simultaneously.

The embodiments described above are fully automatic. In some examples a user or operator of the system may manually instruct some steps of the method to be carried out.

In the described embodiments of the invention the system may be implemented as any form of a computing and/or electronic device. Such a device may comprise one or more processors which may be microprocessors, controllers or any other suitable type of processors for processing computer executable instructions to control the operation of the device in order to gather and record routing information. In some examples, for example where a system on a chip architecture is used, the processors may include one or more fixed function blocks (also referred to as accelerators) which implement a part of the method in hardware (rather than software or firmware). Platform software comprising an operating system or any other suitable platform software may be provided at the computing-based device to enable application software to be executed on the device.

Various functions described herein can be implemented in hardware, software, or any combination thereof. If implemented in software, the functions can be stored on or transmitted over as one or more instructions or code on a computer-readable medium. Computer-readable media may include, for example, computer-readable storage media. Computer-readable storage media may include volatile or non-volatile, removable or non-removable media implemented in any method or technology for storage of information such as computer readable instructions, data structures, program modules or other data. A computer-readable storage media can be any available storage media that may be accessed by a computer. By way of example, and not limitation, such computer-readable storage media may comprise RAM, ROM, EEPROM, flash memory or other memory devices, CD-ROM or other optical disc storage, magnetic disc storage or other magnetic storage devices, or any other medium that can be used to carry or store desired program code in the form of instructions or data structures and that can be accessed by a computer. Disc and disk, as used herein, include compact disc (CD), laser disc, optical disc, digital versatile disc (DVD), floppy disk, and blu-ray disc (BD). Further, a propagated signal is not included within the scope of computer-readable storage media. Computer-readable media also includes communication media including any medium that facilitates transfer of a computer program from one place to another. A connection, for instance, can be a communication medium. For example, if the software is transmitted from a website, server, or other remote source using a coaxial cable, fiber optic cable, twisted pair, DSL, or wireless technologies such as infrared, radio, and microwave are included in the definition of communication medium. Combinations of the above should also be included within the scope of computer-readable media.

Alternatively, or in addition, the functionality described herein can be performed, at least in part, by one or more hardware logic components. For example, and without limitation, hardware logic components that can be used may include Field-programmable Gate Arrays (FPGAs), Program-specific Integrated Circuits (ASICs), Program-specific Standard Products (ASSPs), System-on-a-chip systems (SOCs). Complex Programmable Logic Devices (CPLDs), etc.

Although illustrated as a single system, it is to be understood that the computing device may be a distributed system. Thus, for instance, several devices may be in communication by way of a network connection and may collectively perform tasks described as being performed by the computing device.

Although illustrated as a local device it will be appreciated that the computing device may be located remotely and accessed via a network or other communication link (for example using a communication interface).

The term ‘computer’ is used herein to refer to any device with processing capability such that it can execute instructions. Those skilled in the art will realise that such processing capabilities are incorporated into many different devices and therefore the term ‘computer’ includes PCs, servers, mobile telephones, personal digital assistants and many other devices.

Those skilled in the art will realise that storage devices utilised to store program instructions can be distributed across a network. For example, a remote computer may store an example of the process described as software. A local or terminal computer may access the remote computer and download a part or all of the software to run the program. Alternatively, the local computer may download pieces of the software as needed, or execute some software instructions at the local terminal and some at the remote computer (or computer network). Those skilled in the art will also realise that by utilising conventional techniques known to those skilled in the art that all, or a portion of the software instructions may be carried out by a dedicated circuit, such as a DSP, programmable logic array, or the like.

It will be understood that the benefits and advantages described above may relate to one embodiment or may relate to several embodiments. The embodiments are not limited to those that solve any or all of the stated problems or those that have any or all of the stated benefits and advantages.

Any reference to ‘an’ item refers to one or more of those items. The term ‘comprising’ is used herein to mean including the method steps or elements identified, but that such steps or elements do not comprise an exclusive list and a method or apparatus may contain additional steps or elements.

As used herein, the terms “component” and “system” are intended to encompass computer-readable data storage that is configured with computer-executable instructions that cause certain functionality to be performed when executed by a processor. The computer-executable instructions may include a routine, a function, or the like. It is also to be understood that a component or system may be localized on a single device or distributed across several devices.

Further, as used herein, the term “exemplary” is intended to mean “serving as an illustration or example of something”.

Further, to the extent that the term “includes” is used in either the detailed description or the claims, such term is intended to be inclusive in a manner similar to the term “comprising” as “comprising” is interpreted when employed as a transitional word in a claim.

The figures illustrate exemplary methods. While the methods are shown and described as being a series of acts that are performed in a particular sequence, it is to be understood and appreciated that the methods are not limited by the order of the sequence. For example, some acts can occur in a different order than what is described herein. In addition, an act can occur concurrently with another act. Further, in some instances, not all acts may be required to implement a method described herein.

Moreover, the acts described herein may comprise computer-executable instructions that can be implemented by one or more processors and/or stored on a computer-readable medium or media. The computer-executable instructions can include routines, sub-routines, programs, threads of execution, and/or the like. Still further, results of acts of the methods can be stored in a computer-readable medium, displayed on a display device, and/or the like.

The order of the steps of the methods described herein is exemplary, but the steps may be carried out in any suitable order, or simultaneously where appropriate. Additionally, steps may be added or substituted in, or individual steps may be deleted from any of the methods without departing from the scope of the subject matter described herein. Aspects of any of the examples described above may be combined with aspects of any of the other examples described to form further examples without losing the effect sought.

It will be understood that the above description of a preferred embodiment is given by way of example only and that various modifications may be made by those skilled in the art. What has been described above includes examples of one or more embodiments. It is, of course, not possible to describe every conceivable modification and alteration of the above devices or methods for purposes of describing the aforementioned aspects, but one of ordinary skill in the art can recognize that many further modifications and permutations of various aspects are possible. Accordingly, the described aspects are intended to embrace all such alterations, modifications, and variations that fall within the scope of the appended claims.

1. A computer-implemented method of selecting a cell line for an assay, the method comprising:
encoding data, which is comprised of one or more features, as one or more latent variables;
identifying the one or more features encoded in the latent variables;
mapping the one or more latent variables to cell lines based on the one or more features;
determining a relevance of one or more targets to each of one or more of the one or more latent variables; and
matching the one or more targets to the cell lines via the one or more latent variables.

2. The computer-implemented method of claim 1, wherein the data comprises at least one of genomics data, transcriptomics data, methylation data, clinical data applied to genes, and biological mechanisms associated with multiple features.

3. The computer-implemented method of claim 1, comprising encoding the data as the one or more latent variables using linear and non-linear machine learning models.

4. The computer-implemented method of claim 1, comprising encoding the data as the one or more latent variables using a matrix factorisation approach.

5. The computer-implemented method of claim 1, comprising encoding the data as the one or more latent variables using a clustering algorithm.

6. The computer-implemented method of claim 1, comprising associating a biological mechanism with each latent variable based on the one or more features that the latent variable encodes.

7. The computer-implemented method of claim 1, comprising determining an extent to which each latent variable is associated with a respective biological mechanism.

8. The computer-implemented method of claim 1, comprising removing from consideration the one or more latent variables not sufficiently associated with a biological mechanism.

9. The computer-implemented method of claim 1, comprising assessing the one or more latent variables for relevance to a disease.

10. The computer-implemented method of claim 9, comprising determining an extent to which a respective biological mechanism associated with a latent variable is associated with the disease.

11. The computer-implemented method of claim 9, comprising annotating the one or more latent variables based on relevance to the disease.

12. The computer-implemented method of claim 9, comprising removing from consideration the one or more latent variables not sufficiently relevant to the disease.

13. The computer-implemented method of claim 9, wherein two or more latent variables represent respective biological mechanisms of the disease.

14. The computer-implemented method of claim 1, comprising using the latent variables to stratify patients into endotypes.

15. The computer-implemented method of claim 1, comprising mapping a latent variable to a cell line if one or more features in the cell line sufficiently match the one or more features encoded in the latent variable.

16. The computer-implemented method of claim 1, comprising assigning a mapping value to each latent variable and cell line pair based on a relevance of the cell line to the latent variable.

17. The computer-implemented method of claim 16, wherein the relevance of the cell line to the latent variable is based on an extent to which one or more features in the cell line matches the one or more features encoded in the latent variable.

18. The computer-implemented method of claim 1, comprising determining a relevance score of each target to each latent variable based on the extent to which the target regulates one or more of the genes encoded in the latent variable.

19. The computer-implemented method of claim 1, comprising annotating a respective latent variable with targets that sufficiently regulate one or more of the features encoded in the respective latent variable.

20. The computer-implemented method of claim 1, comprising matching targets to cell lines by comparing the mapping of the one or more latent variables to the cell lines and the relevance of the targets to the one or more latent variables.

21. The computer-implemented method of claim 1, further comprising:
assigning a mapping value to each latent variable and cell line pair based on a relevance of the cell line to the latent variable;
determining a relevance score of each target to each latent variable based on the extent to which the target regulates one or more of the genes encoded in the latent variable; and
for each latent variable, determining a metric between each target and each cell line based on:
the mapping value of the latent variable and the cell line; and
the relevance score of the target and the latent variable.

22. The computer-implemented method of claim 21, wherein the metric is also based on the relevance of the latent variable to the disease.

23. The computer-implemented method of claim 21, comprising outputting a ranked list of cell lines for each target based on the metrics.

24. The computer-implemented method of claim 1, wherein the one or more features comprise one or more genes, methylation sites, and genetic variants; or wherein the one or more features of the cell line comprise gene expression.

25. A computer-readable medium storing code that, when executed by a computer, causes the computer to perform the method of claim 1.

26. A system for selecting a cell line for an assay, the system comprising:
an encoder configured to encode data as one or more latent variables;
an interpretation module configured to identify one or more features encoded in the one or more latent variables;
a cell line mapping module configured to map the one or more latent variables to cell lines;
a target relevance module configured to determine a relevance of one or more targets to each of one or more of the one or more latent variables; and
a matching module configured to match the one or more targets to the cell lines via the one or more latent variables.
